(19) United States
(12) Patent Application Publication
(10) Pub. No.: US 2024/0359012 A1
(43) Pub. Date: Oct. 31, 2024

(54) System and Method for the Automatic Design of Electrical Waveforms for Selective Neuron Stimulation

(71) Applicant: CARNEGIE MELLON UNIVERSITY, Pittsburgh, PA (US)

(72) Inventors: Pulkit Grover, Pittsburgh, PA (US); Chaitanya Goswami, Pittsburgh, PA (US)

(73) Assignee: CARNEGIE MELLON UNIVERSITY, Pittsburgh, PA (US)

(21) Appl. No.: 18/640,353

(22) Filed: Apr. 19, 2024

Related U.S. Application Data
(60) Provisional application No. 63/460,962, filed on Apr. 21, 2023.

(51) Int. Cl. A61N 1/36; G06F 17/11

(52) U.S. Cl. CPC A61N 1/36135; A61N 1/3615; G06F 17/11

(57) ABSTRACT

Disclosed herein is a novel pseudoinverse estimation system and method that adapts regression techniques to directly estimate one or more pseudo inverses of a neuromodulation pathway, thereby circumventing the need of inverting an estimated forward model to design an electrical waveform that elicits a desired neural response. This is accomplished by the learning of a restricted domain that restricts the potential stimuli required to produce the desired neural response. Also disclosed herein is an adaptive, data-driven method providing a more selective design of an electrical waveform to elicit the desired neural response.

### RELATED APPLICATIONS

This application claims the benefit of U.S. Provisional Patent Application No. 63/460,962, filed Apr. 21, 2023. This application is also a continuation-in-part filing under 35 U.S.C. § 365 (c) of PCT application PCT/US2023/078171, filed in the United States Receiving Office on Oct. 30, 2023. The contents of both applications are incorporated herein in their entireties.

### GOVERNMENT INTEREST

This invention was made with U.S. Government support under contract N65236-19-C-8017, awarded by the U.S. Department of the Navy. The U.S. government has certain rights in this invention.

### BACKGROUND OF THE INVENTION

Neuromodulation refers to altering neural activity through the targeted delivery of a stimulus (e.g., electrical, chemical, ultrasound), and is one of the fastest growing areas of medicine, impacting millions of patients. Many neurological disorders and diseases are a result of atypical neural activity in the brain and can be treated by providing appropriate stimuli which can “correct” the atypical neural activity.

In experiments, controlling the neural activity through stimuli has shown promise in treating Parkinsonian symptoms, stroke rehabilitation, regulating depression, etc. The ability to systematically design stimuli that produce a desired neural response in the brain which corrects the atypical activity is key to treating several neurological disorders.

Typically, a stimulus is characterized by a set of parameters. For example, electrical currents (stimuli) can be injected into the brain to selectively stimulate a particular type of neuron (controlling neural activity) for treating Parkinsonian symptoms. This method has been tried in mice subjects, in which the stimulus is characterized by three parameters, namely, amplitude, frequency, and duration of the injected electrical currents.

A common way to mathematically model the relation between the parameters of stimuli and the neural activity/response is through a “forward mapping/function” that takes as input the parameters and outputs the neural response. The problem of designing a stimulus that produced the desired neural response is then reduced to inverting the forward mapping. Correspondingly, the set of stimulus parameters can be obtained by plugging in the desired neural response as an input to the inverse.

Broadly, there are three major challenges in estimating the inverse of the forward mapping. First, as the forward mapping depends upon the parameters being explored, for novel parameters, the forward mapping is generally unknown and needs to be estimated from the data. Second, in most cases of interest, multiple sets of parameter values characterizing a stimulus lead to the same neural response. For example, in electrical stimulation of the brain, many stimuli produce the same neural firing rate and consequently, the same neural response. This implies that the forward mapping in many cases is many-to-one, and hence, non-invertible. Therefore, instead of estimating an inverse, a pseudoinverse of the forward mapping is estimated. Third, the amount of data available in such healthcare settings is limited, and in general, the data collection process is quite expensive.

It would be desirable to be able to estimate the pseudoinverse in a data-efficient manner. There has been a significant interest in designing stimuli using data-driven method through some form of pseudoinverse estimation. A main advantage of data-driven approaches over traditional stimulus design approaches is that they allow end users (e.g., clinicians or neuroscientists) to explore a larger number of parameters as compared to traditional approaches. The ability to explore a large number of parameters allows the possibility of discovering novel stimuli that can produce neural responses which have clinical relevance.

Most current approaches to waveform design for selective neuro stimulation are either model-based, therapy limiting their practical applicability, and/or use trial and error, which allows only a limited exploration of the waveform space. Therefore, it would be desirable to ne able to design to selectively stimulate different neuron types using a data-based approach which does not rely on a model or trial and error.

### SUMMARY OF THE INVENTION

Disclosed herein in a first embodiment of the invention is a novel pseudoinverse estimation system and method that addresses the shortcomings of the two methodologies mentioned above. Specifically, the disclosed method adapts regression techniques to, in one embodiment, directly estimate a pseudoinverse, thereby circumventing the need of inverting an estimated forward model, while still requiring less data than CDE methods (because regression techniques typically require less data than their equivalent CDE counterpart). In a second aspect of the first embodiment, an ensemble of pseudoinverses is estimated.

The key insight of the disclosed method is that a non-invertible function can still be inverted over a restricted domain. If such a restricted domain is known a priori, the inverse mapping can be estimated using regression methods. The disclosed method jointly learns a restricted domain and the inverse mapping over it. A weighted L2 loss is utilized, where the weights are also learned from data. On convergence, the weights approximate the indicator function over the restricted domain, effectively learning it.

When compared with other methods, for example, Masked Autoregressive Flows (MAF) and Mixture Density Networks (MDN), the disclosed method outperforms all the other methods when the size of dataset is small.

In a second embodiment of the invention, a novel method for designing electrical waveforms for achieving selective stimulation among different neuron types which relies directly on data, without relying on the assumption of a computational model is disclosed. A major hurdle for data-driven approaches is that only a small amount of data can be sampled to design waveforms (e.g., ˜250 datapoints). Hence, any viable data-driven technique must necessarily be data-efficient.

The second embodiment of the invention discloses a method to adaptively design a selective waveform in a data efficient manner. The disclosed method of the second embodiment is a data-driven adaptive algorithm, in which all previously-collected data informs the estimation of a more selective waveform. This allows the disclosed method to find waveforms exhibiting higher selectivity by exploring only a few datapoints, thereby making it data efficient.

### BRIEF DESCRIPTION OF THE DRAWINGS

FIG. 1 are graphs showing the normalized mean absolute error (NMAE) of the

disclosed method versus various prior art data-driven methods discussed herein on toy mappings.

FIG. 2 are graphs visualizing pseudoinverses for toy mappings.

FIG. 3 show graphs showing the NMAE across both neuron models with different training datasets sizes constructed from bio-physical computational neuron models.

FIG. 4 is a schematic representation of the waveforms used in the simulation study.

FIG. 5 is a flow chart of the process of the first embodiment of the invention.

FIG. 6 is a data flow diagram of the process of the first embodiment of the invention.

FIG. 7 is a flow chart of the process of the second embodiment of the invention.

### DETAILED DESCRIPTION

Disclosed herein as a first embodiment is a method for directly estimating a pseudoinverse neural mapping to affect a desired neural response by restricting the domain over which the forward mapping from the stimulus to the neural response is inverted.

Assume that each stimulus is characterized by n-different parameters, denoted as {θᵢ}ᵢ₌₁ⁿ, where θᵢ∈ [symbol] . Define θ=[θ₁, θ₂, . . . , θₙ]^{T} ∈ [symbol] ⁿ as the collection of all the n parameters. Θ⊂ [symbol] ⁿ denotes the collection of all allowed stimuli. Let the number of neural responses of interest be m (e.g., for selective stimulation between two neuron types, m=2). Define r=[r₁, r₂, . . . rₘ]^{T}∈ [symbol] ^{m}. Let [symbol] _{Θ} be the collection of all distinct neural responses produced by all the stimuli θ∈Θ.

To illustrate the notation further, consider an example in which the goal is to selectively stimulate a single neuron type without stimulating another neuron type for treating Parkinsonian symptoms. The stimuli of electrical currents is characterized by 3 parameters which are the amplitude, frequency and duration of the currents. So, in this example the parameters would be θ₁, θ₂, θ₃ respectively denoting the amplitude, frequency and duration of the electrical currents, and θ=[θ₁, θ₂, θ₃]^{T}∈ [symbol] ³. The relevant neural response in the same example are the firing rates of the different neuron types, so r=[r₁, r₂]^{T}, where r₁ and r₂ are the firing rates of the two neuron types. For finding a stimulus that attains a desired neural response, assume access to a dataset consisting of only [symbol] =[θᵢ, rᵢ]ᵢ₌₁^{N} formed by N stimulus-response pairs.

Problem Statement-Given a dataset [symbol] where {θᵢ}ᵢ₌₁^{N} are independent and identically distributed samples from a distribution [symbol] (θ) on Θ, and rᵢ is the neural response generated by the stimulus characterized by θᵢ, the goal is to design/find a parameter θ_{des}∈ [symbol] such that the stimulus corresponding to θ_{des} can elicit (close to) a desired (user-specified) neural response r_{des}∈ [symbol] _{Θ}.

Note that access is only allowed to the fixed dataset [symbol] . An important design parameter is choosing the set of allowed stimuli (i.e., Θ). This is decided a priori by the user, typically based on domain knowledge, (e.g., choose amplitude, frequency, and duration of the electrical waveforms as parameters of stimuli). Herein, it is assumed that that an appropriate Θ has already been chosen.

Denote the forward mapping from the stimulus parameter space Θ to the neural response space [symbol] _{Θ} as g: Θ→ [symbol] _{Θ}. For many-to-one functions such as g, a natural definition of a pseudoinverse is a mapping g⁻¹: [symbol] _{Θ}→Θᵢₙᵥ, where Θᵢₙᵥ⊆Θ is a restricted domain such that g(g⁻¹(r))=r∀r∈ [symbol] _{Θ}. For example, cos: [symbol] →[−1,1] is many-to-one and not invertible but restricting the domain of cos to [0, π] helps define the familiar pseudoinverse: cos⁻¹: [−1,1]→[0, π]. Note that, for a pseudoinverse, equality in the other direction, g⁻¹(g(θ))=θ∀θ∈Θ is not necessarily true, because the domain of g⁻¹ is Θᵢₙᵥ (a subset of Θ). A function can have multiple pseudoinverses (e.g., cos(⋅), with Θᵢₙᵥ=[0+2nπ, π+2nπ], has infinitely many pseudoinverses, one for each n∈N. For the goal of the method disclosed herein, estimating any one pseudoinverse suffices. The estimated pseudoinverse of g is denoted as [symbol] herein.

The Method—The disclosed method estimates a pseudoinverse by exploiting the insights that many-to-one functions can be inverted over an appropriately restricted domain. If such a restricted domain Θᵢₙᵥ was known a priori, then a restricted dataset can be created by excluding all data points (θᵢ, rᵢ) where θᵢ∉Θᵢₙᵥ from the dataset [symbol] . Because g is invertible over Θᵢₙᵥ, any traditional regression technique applied to this restricted dataset would yield a pseudoinverse corresponding to Θᵢₙᵥ. Formally, a pseudoinverse on Θᵢₙᵥ can be estimated as:

$$= arg {min}_{f ∈ ℱ} \frac{1}{N} {\sum }_{i = 1}^{N} 𝕀 [ {θ}_{i} ∈ {Θ}_{i n v} ] {‖ f ( {r}_{i} ) - {θ}_{i} ‖}_{2}^{2} \qquad ( 1 )$$

where:
[symbol] (⋅) is the indicator function; and
[symbol] is the family of functions being considered for regression.

The challenge, as outlined above, is that only the dataset (and not the forward mapping) can be accessed, so Θᵢₙᵥ is not known a priori. To address this, the disclosed method jointly estimates both a restricted domain and the corresponding pseudoinverse as follows:

$$, {\{ \hat{w} ( {θ}_{i} ) \}}_{i = 1}^{N} = arg {min}_{\begin{matrix}f ∈ ℱ , \\ w ( {θ}_{i} ) \geq 0\end{matrix}} {\sum }_{i = 1}^{N} {\underbrace{w ( {θ}_{i} ) {‖ f ( {r}_{i} ) - {θ}_{i} ‖}_{2}^{2}}}_{Loss} + {\underbrace{β {\sum }_{i = 1}^{N} {w}^{2} ( {θ}_{i} )}}_{Regularizer} , \qquad ( 2 )$$

$$s . t . \frac{1}{N} {\sum }_{i = 1}^{N} w ( {θ}_{i} ) = 1$$

where:
β∈ [symbol] ⁺ is a hyper-parameter.

The first sum in Eq. (2) represents the loss, and the second sum in Eq. (2) is a regularizer. This optimization formulation follows the philosophy of Eq. (1), approximating [symbol] [θᵢ∈Θᵢₙᵥ] in Eq. (1) by ŵ(θᵢ) which are learned jointly with [symbol] .

How does the optimization of Eq. (2) incentivize learning of a restricted domain? If only parameters belonging to a restricted domain have non-zero weights (ŵ(θᵢ)), the loss term would be low because the corresponding inverse mapping can be estimated accurately. Hence, the loss term encourages the learning of weights that are non-zero only for some restricted domain over which g is invertible.

It is desirable that an estimate of the pseudoinverse [symbol] has its domain as the entire [symbol] _{Θ}, or at least as large a subset as possible, so it can provide a neural stimuli parameter for as many neural responses as possible. This implies that, for the disclosed method to estimate a pseudoinverse, the image of the restricted domain learned by it should be as large as possible. This condition is not ensured by the loss term in Eq. (2), since it is small for any restricted domain (e.g., consider two restricted domains [0,1] and [0, π] of cos(⋅). [0, π] is more desirable here. The loss term in Eq. (2) is small for both domains and is unable to discriminate between them).

To encourage the disclosed method to learn a restricted domain corresponding to a large response range/space, we use the following observation: If Θₘₐₓ is the restricted domain having the largest size (measured by its total probability under p(θ)), then it's corresponding image/response space also has the largest size. Hence, by learning the largest restricted domain we ensure we have the largest response space. This observation is incorporated in the regularizer and the constraint in Eq. (2) to encourage the method to learn as large a restricted domain as possible. To see this, analyze the following optimization that distills the effect of the regularizer for distinguishing among restricted domains over which g is invertible (the first term in Eq. (2) is low for such domains):

$${\{ {w}_{i}^{*} \}}_{i = 1}^{N} = arg {min}_{{\{ {w}_{i} \}}_{i = 1}^{N}} {\sum }_{i = 1}^{N} {w}_{i}^{2} , s . t . \frac{1}{N} {\sum }_{i = 1}^{N} {w}_{i} = 1 , \qquad ( 3 )$$

$${\sum }_{i = 1}^{N} 𝕀 [ {w}_{i} \neq 0 ] = K , {w}_{i} \geq 0 ∀ i$$

This optimization explores the behavior of the regularizer when only K out of N total weights are non-zero and has the solution: w*ᵢ=N/K for any K out of N weights, and the rest are 0. The regularizer term in Eq. (2) scales approximately as ˜1/K² for K non-zero weights, which incentivizes making larger numbers of ŵ(θᵢ) to be non-zero, encouraging the consideration of as large a restricted domain as possible.

Thus, there is a careful interplay between the loss, the regularizer and the constraint in Eq. (2). The loss encourages learning non-zero weights (ŵ(θᵢ)) only over a restricted domain; the regularizer and the constraint try to make the restricted domain as large as possible. By carefully choosing the value of B, a desirable pseudoinverse can be learned.

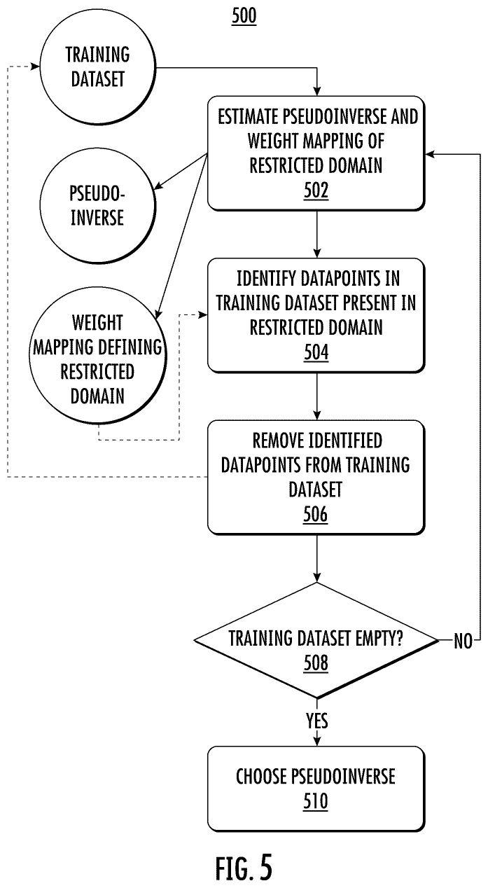

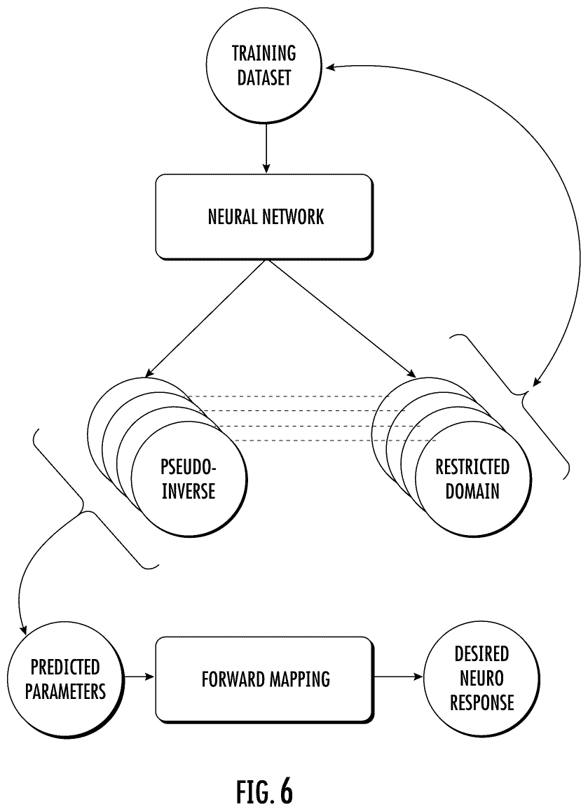

In a second aspect of the first embodiment of the invention, an extension of the disclosed method estimates an ensemble of pseudoinverses, instead of just one, thereby improving the performance. Process 500 of the second aspect of the first embodiment is shown in flowchart form in FIG. 5. The process 500 shown in FIG. 5 creates an ensemble of pseudoinverses in a greedy fashion as follows: (i) the method as previous discussed is used to estimate the pseudoinverse and the corresponding weight mapping on the training dataset at step 502; (ii) at step 504, the estimated weight mapping is used to identify the datapoints in the training dataset that lie in the restricted domain (estimated in step (i)). At step 506, the identified data points are removed from the training dataset to construct a new training dataset; and (iii) This new training dataset is then again used to perform steps 502 and 504 (i.e., (i) and (ii) above, respectively). This process continues until the (remaining) training dataset becomes empty at step 506, resulting in an a plurality of pseudoinverses and corresponding restricted domains that are distinct due to step (ii). To predict the parameter vector 0 for some response vector, the pseudoinverse whose restricted domain contains the training datapoint with the response vector closest to the desired response vector output is chosen at step 510. Intuitively, this pseudoinverse would have the most confidence in predicting the right parameter. Note that no prior art method creates an ensemble of pseudoinverses in this fashion, as only the ensemble embodiment of the disclosed method explicitly estimates the restricted domain (in terms of the weight mapping) which is required for step (ii). A data flow diagram of the process is shown in FIG. 6. Although only four pseudoinverses and corresponding restricted domains are shown, as would be realized, any number of pseudoinverses and corresponding restricted domains could be produced by process 500.

Simulations—Two scenarios were simulated-toy examples and bio-physical neuron models, in which the performance of the disclosed method as well as 3 prior art data-driven methods (MAF, MDN and Naïve Inversion (NI)) were compared for estimating pseudoinverses (i.e., [symbol] (⋅)) In addition, a baseline approach was implemented.

BASELINE—In addition to the data-driven methods, a brute force approach, akin to the 1-Nearest Neighbor Method, is used as a baseline method for the simulation study performed. In the baseline method, the training data is used as a look-up table, where we output the parameter vector present in the training dataset whose response vector is closest to the desired response vector under the L1 distance metric.

SIMULATION WITH TOY EXAMPLES: Three different toy mappings were considered for the purpose of estimating pseudoinverses. The following models were used for generating the data from each toy mapping:

$$r = cos ( 2 π θ ) + ϵ , θ = [ 0 , 3 ] \qquad ( 4 )$$

$$r = {( {θ}^{2} - 4 )}^{2} + ϵ , θ = [ - 3 , 3 ] \qquad ( 5 )$$

$$r = {e}^{- \frac{{θ}^{2}}{2}} + ϵ , θ = [ - 3 , 3 ] \qquad ( 6 )$$

where:
ϵ˜ [symbol] (0,0.01) is the Gaussian distribution with mean 0 and variance 0.1\hat{ } 2 for cos(⋅) toy examples, 2.5\hat{ }2 for the polynomial(⋅) example and 0.2\hat{ }2 for the exponential example. In the small data regime, the disclosed method outperforms MAF, MDN and NI in estimating the pseudoinverse.

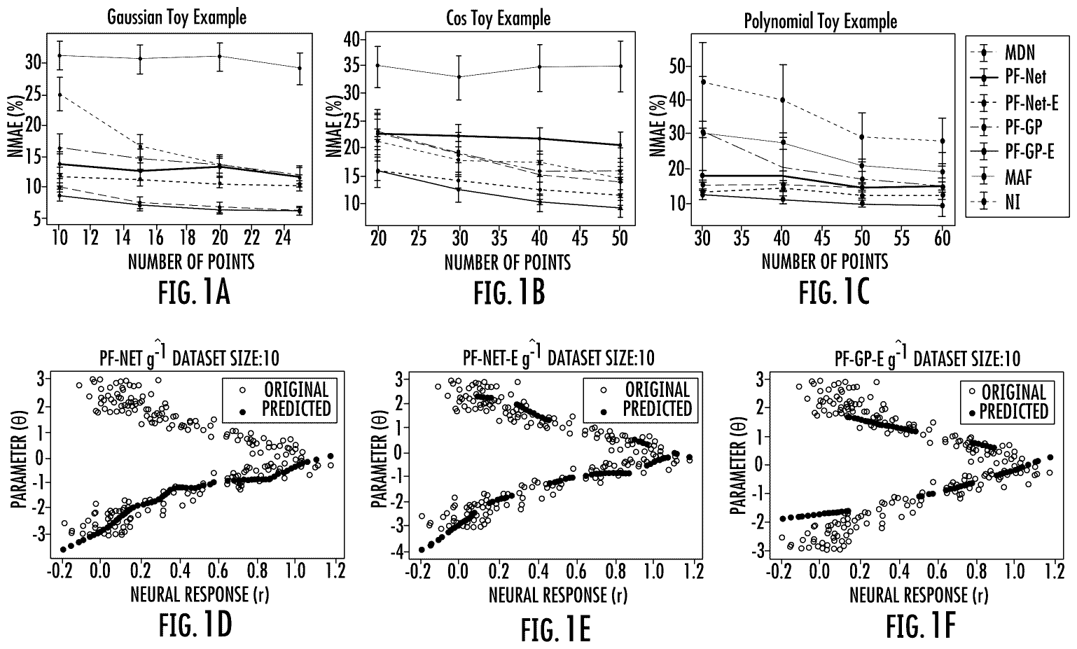

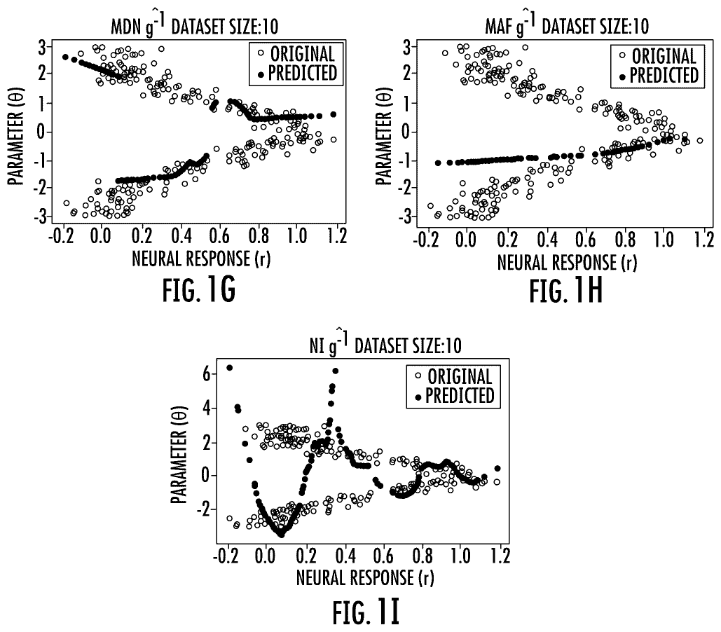

To characterize the data-efficiency of each technique, (i.e., the four variants of the disclosed method and MDN, MAF, NI) in estimating the pseudoinverses of the above toy mappings, the following study was performed: For each toy forward mapping we evaluate the NMAE at 4 different training dataset size. The resultant NMAE for all the four techniques and the three different toy mappings are shown in FIG. 1.

It can be seen a variant of the disclosed method (denoted as “PF-NET”, “PF-NET-E”, “PF-GP” or “PF-GP-E” in the FIGS. 1(a-c) has the smallest NMAE among all the competing techniques consistently across the three different toy forward mappings. This provides evidence that the method requires the least amount of data to estimate pseudoinverse with reasonable accuracy. This result extends to the more realistic simulation study of electrical stimulation of neurons below.

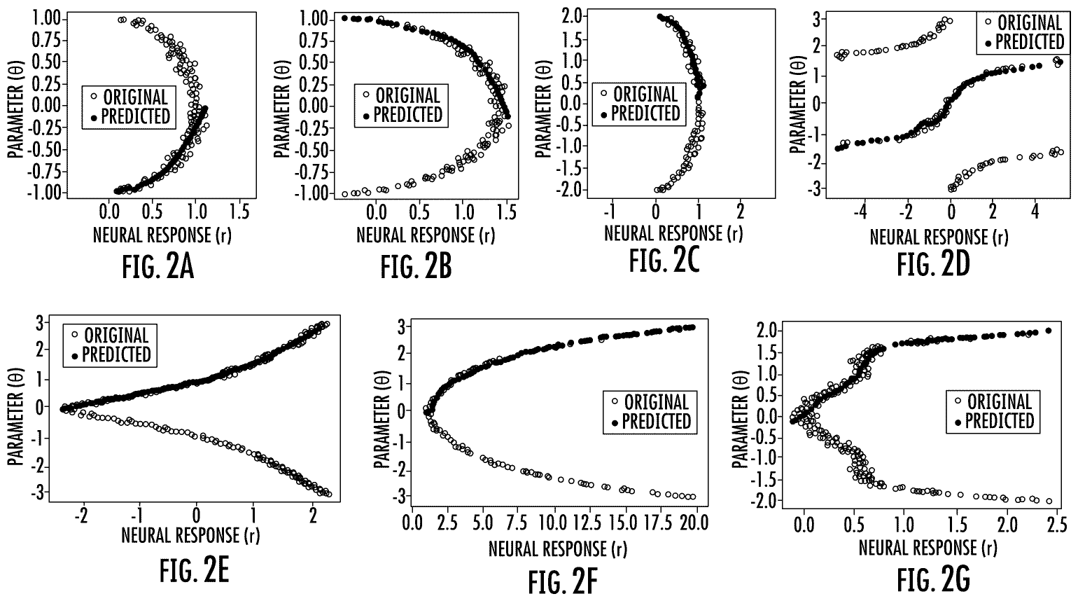

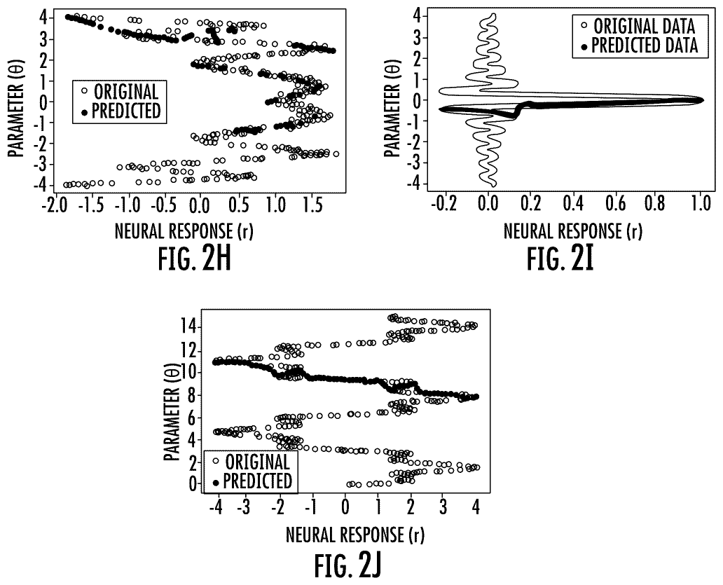

FIG. 2 shows graphs visualizing pseudoinverses estimated by the disclosed method for several non-linear functions. The corresponding forward functions for each subfigure are as follows: FIG. 2a:

$$r = \sqrt{1 - {θ}^{2}} ;$$

### FIG. 2b:

$$r = 2 \sqrt{1 - {θ}^{2}} ;$$

### FIG. 2c:

$$r = \sqrt{1 - {( θ / 2 )}^{2}} ;$$

FIG. 2d: r=tan θ; FIG. 2e: r=log(0.1+x²); FIG. 2f: r=e^{|x|}, FIG. 2g: r=x²+cos(x²)−1: FIG. 2h: r=sin(|x|)+cos (x²); FIG. 2i:

$$r = sin ( π x ) / ( π x )$$

and FIG. 2j: r=3 sin(θ)+sin(5θ).

SIMULATION WITH NEURON MODELS—The disclosed method and the 3 competing

prior art models (MDN, MAF and NI) were applied to estimate the parameters of stimulus (electrical waveform in this particular case) for desired neural responses (firing rate of the two neuron models).

The main goal of this simulation is to show that the performance of the disclosed method is better at “small” dataset size and is comparable to other methodologies at “large” dataset size. That is, qualitatively the results obtained for toy examples continue to hold in a more realistic scenario.

The simulation evaluates the ability of electrical waveforms to stimulate a bio-physically detailed cortical neuron model of excitatory pyramidal cell (Pyr neurons) and inhibitory parvalbumin-expressing inhibitory neurons (PV neurons). The ability to modulate the activity/firing rate of excitatory and inhibitory neurons using electrical currents has clinical relevance.

The NMAE was calculated for all the data-driven approaches, as well as the baseline approach and the results are shown in FIG. 2. For disclosed method, only the results for the best performing variant across all the toy examples are shown. FIG. 2a. shows the mean of the NMAE across the two neuron types, for all the methods with different training dataset size constructed from the bio-physical computational neuron models. FIG. 2b. is a zoomed-in version of FIG. 2a showing the average NMAE for the three top-performing methods, namely the disclosed method, Baseline, and MAF. The values of NMAE at each training dataset size and for each technique was calculated across 50 different trials, and the average NMAE is reported. The color bars show the 95% confidence interval. FIG. 2c shows the relative decrease in the average NMAE of the disclosed method compared to the next-best alternative method. To quantify this relative decrease, we plot (NMAE of next best alternative NMAE of the disclosed method)/NMAE of the disclosed method×100 for every training dataset size. The particular dataset sizes investigated for the neuron model case are informed by experimental constraints in collecting datasets.

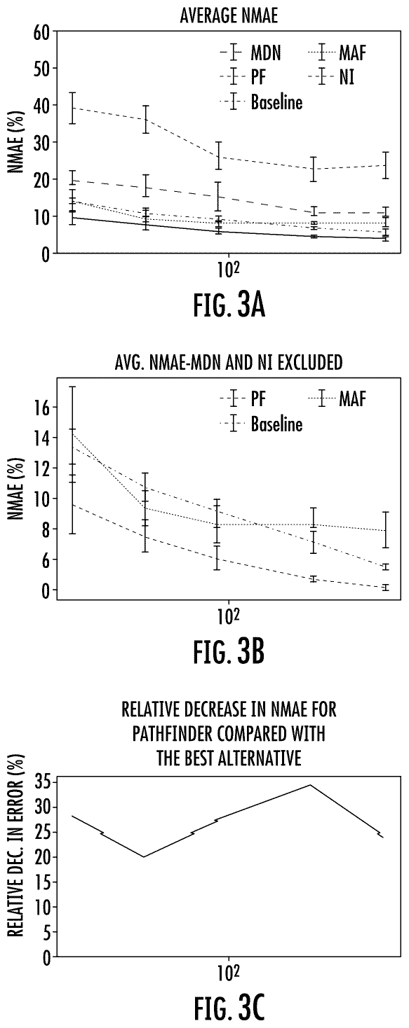

As with the toy examples, it was observed that the disclosed method significantly outperforms the other techniques for low sample size, as shown in FIGS. 3(a-c). The disclosed method has >20% less NMAE compared to the best alternative for all training dataset sizes (FIGS. 2a, 2b, and 2c.). Notably, disclosed method requires only 50 datapoints to achieve the performance of the best alternative method at at 250 datapoints.

To create a dataset of size N for a toy mapping (i.e., [symbol] _{toy}={θᵢ,rᵢ}ᵢ₌₁^{N}), N samples of θ were uniformly randomly sampled from the toy forward mapping's respective domains provided in Eqs. (4-6). The corresponding {rᵢ}ᵢ₌₁^{N} for each toy mapping were then generated according to the respective models provided in Eqs. (4-6). Different values of N were considered for different toy mappings to compare performance of the disclosed method and the three prior art methods across different dataset sizes.

Bio-physical Neuron Models: It is well known that cortex consists of excitatory and inhibitory neurons, and it is hypothesized that a careful interplay between the activation of excitatory and inhibitory neurons in the cortex drives the neural activity in the cortex. Imbalance in the activation of excitatory and inhibitory neurons is linked to neurological diseases such as seizures, hence being able to modulate the activity of excitatory and inhibitory neurons is of clinical relevance. In recent years, there has been a growing interest in exploring electrical stimulation as a means to stimulate excitatory and inhibitory at desired firing rates. Controlling the firing rates of excitatory and inhibitory neurons (neural response) with electrical stimulation (stimulus) provides a good simulation setup to test the performance of the disclosed method and the three prior art methods. in a more realistic neuromodulation context.

Morphologically-realistic and bio-physically detailed multicompartment neuron models taken from the Allen Cell Type Database were used in the simulations. One neuron model is of a cortical pyramidal (Pyr; excitatory) neuron and the other is of a cortical parvalbumin-expressing (PV; inhibitory) neuron. Both neurons were simulated using the NEURON software, with the allensdk package in python.

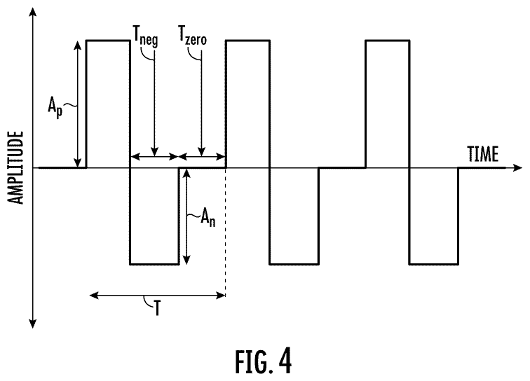

A parametric waveform family using 5 parameters is constructed: Q, Aₙ/Aₚ, T, T_{zero}/T, T_{neg}/T−T_{zero}, where Aₚ, Aₙ, T, T_{zero}, T_{neg} are as depicted in FIG. 4. Q refers to the total absolute charge of the waveform. The range of each parameter is as follows: [0.15 nC, 0.35 nC] for Q, [0.5, 2] for Aₚ/Aₙ, [0.1 ms, 500 ms] for T, [0, 0.5] for T_{zero}/T, and [0, 0.6] for T_{neg}/T−T_{zero}. Each parameter is uniformly sampled from its respective range. The duration of the waveform is 1000 ms. The corresponding waveform

$${u}_{{\vec{θ}}_{n}} ( t )$$

(generated from \vec{θₙ}) is injected intracellularly into the soma of each neuron model. Synaptic noise is also added to the models by adding additive white Gaussian noise with a mean of zero and variance of 2.25 (nA)² to the input waveform. A peak detection algorithm is run (present in the scipy package in python), on the time-trace of the resulting neuron's membrane potentials to calculate the number of neural spikes. A simplistic definition of the firing rate for this simulation is used:

$$r = \frac{Total Number of Spikes}{1000 ms} \qquad ( 8 )$$

where:
r denotes the firing rate of the neuron model.

The firing rates for both neuron models were collected to construct the neural response rᵢ=[rᵢ₁, rᵢ₂]^{T} (rᵢ₁ for the Pyr neuron and rᵢ₂ for the PV neuron). The range of firing rates in the dataset for the Pyr neuron is [0 Hz, 40 Hz] and for the PV neuron is [0 Hz, 100 Hz].

Because the input to the model is the neural response \vec{rₗ} and not the stimulus parameter \vec{θₗ}, the data is split into training, test and validation sets in the following manner: Split all possible neural responses present in [symbol] into training, validation and test firing rates. Let [symbol] _{V}, [symbol] _{Tr}, [symbol] _{Te} be the sets containing the validation, training and test neural responses. Remove all the (\vec{θₗ}, \vec{rₗ}) from the original dataset [symbol] where \vec{rₗ} is present in the test and validation set, to construct the training dataset D_{Tr}. Note that for any i present in the test set or the validation set, there may be multiple stimulus parameter vectors \vec{θ} in the original dataset which produce F and all of them are removed. The validation dataset [symbol] _{V} can be constructed similarly by removing (\vec{θₗ}, \vec{rₗ}) from the original dataset [symbol] where \vec{rₗ} is present in the training set or the test set. For the test set, only the neural responses are stored (i.e., [symbol] _{Te}), to generate stimuli which produce those neural responses.

After training, each approach outputs a parameter \hat{θ} corresponding to every r. For the disclosed method, \vec{\hat{θ}} is the output of the estimated pseudoinverse, when provided with the input \vec{r}. For the first embodiment, \vec{\hat{θ}} is obtained as the output of the estimated pseudoinverse, when provided with the input \vec{r}. For the embodiment producing the ensemble of pseudo inverse functions, the prediction scheme described above is used.

A figure of merit is used to evaluate the validation/test loss. The figure of merit is explained by taking an example of calculating it over the validation set. For every neural response \vec{r} present in [symbol] _{V}, obtain the corresponding \vec{\hat{θ}}, which is then fed to the neuron/model to obtain its actual firing rate \vec{r}^{act}. That is, g( [symbol] (\vec{r}))=\vec{r}^{act}.

Because there are m different neural responses, a NMAE is calculated for each individual neural response. Let the maximum and minimum values that can be achieved for j^{th} neural response be denoted as: rⱼ^{max}, rⱼ^{min}, the NMAE is defined for the j^{th} neural response as:

$$N M A E = \frac{100}{{N}_{V} ( {r}_{j}^{max} - {r}_{j}^{min} )} {\sum }_{\vec{r} ∈ {ℛ}_{V}} \vert {r}_{j} - {r}_{j}^{a c t} \vert \qquad ( 9 )$$

where:
   |(⋅)| is the absolute function;
N_{V} is the number of response vectors present in the validation set; and
rⱼ and rⱼ^{act} are the j^{th} components of \vec{r} and \vec{r}^{act} respectively.

Note that for calculating NMAE, it is not necessary to explicitly know the restricted domain over which pseudoinverse has been estimated. The NMAE quantifies how close the neural response produced by the predicted stimulus is to the desired neural response on a scale of 0 to 100. The test NMAE can be calculated in a similar manner, where we replace the validation set by the test set. Notice that, for calculating the NMAE, access to the neuron is required (hence the need for accessing the neuron during hyper-parameter tuning.

Implementation-The details of one possible implementation of the disclosed method will now be discussed. To choose the hyper-parameters for all the data-driven approaches, the 10-fold cross-validation NMAE is used. Specifically, the hyper-parameter with the lowest cross-validation NMAE is picked. To calculate the 10-fold cross-validation NMAE, a 90%-10% split of the dataset is created, where 90% of the datapoints are in the training data and 10% of the datapoints are in the validation data, and the NMAE is calculated for the validation data. This process is repeated 10 times, and the average validation NMAE is used as the cross-validation NMAE. An upper and a lower bound of the range for each hyperparameter's search is obtained through a random search, followed by a grid search within these bounds.

Four different variants of the disclosed method were used, referred to herein as PATHFINDER-NET, PATHFINDER-NET-E, PATHFINDER-GP, and PATHFINDER-GP-E. In PATHFINDER-NET and PATHFINDER-GP, the inverse mapping ( [symbol] ) is estimated using a multi-layer perceptron (MLP) and Gaussian Process Regression, respectively. PATHFINDER-NET-E and PATHFINDER-GP-E are, respectively, the ensemble versions of PATHFINDER-NET and PATHFINDER-GP. To estimate the weight mapping (ŵ) for all 4 variants, a MLP is used. To implement the Gaussian Process Regression (GP) in PATHFINER-GP and PATHFINDER-GP-E, an existing implementation was used with default parameters. In the toy examples, all of the MLPs used consist of 1-20 hidden layers, each with 10 hidden units, and GP's covariance kernel was chosen as a weighted summation of the Radial Basis Kernel, White Kernel and Dot Product Kernel. The weights for each kernel are automatically learned in GP by using cross-validation loss. For the neuron model, MLPs with 5 to 8 hidden layers were used, with each layer having 100 hidden units (although the hidden layers could have any number of hidden units), for the regressor (in PATHFINDER-NET and PATHFINDER-NET-E) and weight network for all the 4 variants. The Kernel for the neuron model case was the summation of the Radial Basis Kernel, White Kernel, Rational Quadratic and Dot Product Kernel. The output layer of the MLP for the regressor and the weight network has linear activation. For the toy examples, β was chosen as 0.001 for the cos 2πθ mapping, 0.001 for the

$${e}^{- \frac{{θ}^{2}}{2}}$$

mapping and 0.00001 for the (θ²−4)² mapping. For the neuron model the following values of β: 0.5, 0.1, and 0.05 were searched.

In addition to the data-driven methods, a brute force approach, akin to the 1-Nearest Neighbor Method, is used as a baseline method for the simulation study. In the baseline method, the training data is used as a look-up table, where the parameter vector present in the training dataset whose response vector is closest to the desired response vector under the L1 distance metric is output.

In one embodiment, Rectified Linear Unit (ReLU) activation is used for hidden layers of all approaches. MAF and MDN were trained by minimizing the negative log-likelihood (NLL). Alternatively, other activations, such as tan h, sigmoidal, exponential leaky unit and linear activation could be used.

Both the weight and the regressor were trained simultaneously by minimizing the loss in Eq. (2). During training, a simplex projection was performed on the batch output of the weight network to ensure the constraints specified in Eq. (2) are met). An Adam optimizer with a learning rate of 0.001 is used for minimizing the objectives for all techniques. Batch normalization was applied wherever applicable. Because all the methods were trained for multiple different sizes of the training datasets, batch size for Adam was chosen according to the training dataset size. Each model was trained until the validation loss converged (note loss instead of MAE), defined as a relative change of less than 0.1% in the validation loss between successive steps. Validation loss here was calculated using the more traditional method.

A potential reason for the data efficiency of the disclosed method is the maximization bias. The maximization bias refers to the phenomenon that the method tends to estimate the pseudoinverse with the largest number of datapoints. For example, in the cos(⋅) mapping, assume that the method only estimates 1 of the 6 pseudoinverses corresponding to the restricted domains [0, 0.5], [0.5, 1], [1, 1.5], [1.5, 2], [2, 2.5] and [2.5, 3]. Let nᵢ be the number of datapoints that lie in the restricted domain of the i^{th} pseudoinverse. Recall that the regularizer loss tries to give non-zero weights to as many datapoints as possible. Consequently, the regularizer encourages the choosing of the restricted domain with the largest number of datapoints (i.e., maxᵢ nᵢ. For a size-n dataset sampled using a uniform distribution,

$$𝔼 [ {max}_{i} {n}_{i} ] = \frac{n}{6} + c \sqrt{n} ,$$

where [symbol] (⋅) is the expectation, and c is a constant). Note that, while the expected number of datapoints in any one restricted domain is n/6, the largest restricted domain has extra c\sqrt{n} datapoints. The method loss encourages inversion in just this restricted domain, and thus, is able to harness these extra datapoints, lowering the required overall sample size. On the other hand, the prior art methods, in one way or another, try to estimate the entire forward mapping, and are not designed to harness maximization bias.

The first embodiment is based on data-driven techniques for designing stimuli that produce desired neural responses. Traditionally, such stimuli are designed by choosing 1 or 2 “important” parameters (based on the intuition from the bio-physics of the system) and then brute-force searching across the 1 or 2 parameters to characterize the neural response. For example, one method considers the frequency of the sinusoidal stimulation as a parameter and brute-force searches across the frequency to characterize the neural response. A limitation of such brute-force search approaches is that they do not scale well as the number of parameters increases.

The most important advantage of the data-driven techniques is that allow exploration of a larger number of parameters. The Brute-force techniques require 250 datapoints to achieve the same performance as the disclosed method at 50 datapoints. Hence, the disclosed method allows an exploration of a larger number of parameters due to its data-efficiency. In practice, to an end-user, this implies that instead of choosing 1 or 2 most relevant parameters, they can now choose 5 or 10 most relevant parameters to search across. The advantage of exploring a larger number of parameters is that novel stimuli can be discovered which can produce neural responses which would be impossible to produce by just exploring 1 parameter. Intuitively, data-driven techniques are able to scale well to a higher number of parameters because they exploit the inherent smoothness of the neural response, in one form or other, and provide a more principled way of searching the parameter space.

The data-requirement for these data-driven techniques can be even further reduced by using adaptive sampling techniques. For example, the weight mapping of the disclosed method can be used for sampling only from the restricted domain thereby reducing the data requirements or the estimated p(\vec{θ}|\vec{r}) in the conditional density methods can be used to adaptively sample around a desired response \vec{r}_{des}. The characterization of the data-efficiency of the adaptive-sampling techniques is not addressed herein.

Also disclosed as a second embodiment of the invention is an adaptive data-driven method and model for designing an electrical waveform to produce a desired neural response. Data-driven techniques allow end-users, such as clinicians and neuroscientists, to explore a larger number of data points (i.e., parameters of an electrical waveform) as compared to brute force search methods that allow only a 1 or 2 dada point search space. Exploring a larger number of data points helps in designing novel stimuli that produce neural responses which are clinically relevant.

It is desirable to make data-driven techniques as data-efficient as possible to increase their applicability in different neuromodulation contexts, as collecting data in neuromodulation domain is typically expensive. Towards increasing the data-efficiency of data-driven techniques, the disclosed method is capable of designing stimuli for desired neural responses by iteratively estimating a pseudo inverse function. Data-driven techniques provide an alternative to traditional brute-force search of parameters and can help in designing novel stimuli.

The second embodiment of the invention directly estimates a waveform that will provide selective stimulation of certain types of neurons, while minimizing the stimulation of all other neuron types. Assume that the j^{th} neuron type (j=1, . . . , M), is a black box which, when provided with an input waveform u(⋅), a function of time, outputs a firing rate rⱼ∈ [symbol] . The search for input waveforms is restricted to a parametric family denoted by [symbol] _{Θ}={u_{↓}}_{θ∈Θ}, where Θ⊆ [symbol] ^{K}. Let rⱼ denote the firing rate (i.e., the ratio of the total number of spikes and the time for which the waveform is supplied) of the j^{th} neuron type. The vector r=[r₁, . . . , r_{M}]^{T} is the “firing rate vector”, and the collection of all r's produced by waveforms u_{θ}(t)∈ [symbol] _{Θ} is [symbol] _{Θ}. For designing selectively stimulating waveform, access is only available to samples of the form {θ,r}.

Therefore, the goal of the second embodiment is to design a j^{th} neuron selective waveform uⱼ^{sel}∈ [symbol] _{Θ} that maximizes the firing rate rⱼ of neuron j while keeping the firing rate of all other neuron types below a pre-specified threshold r_{low} (i.e., rᵢ≤R_{low}) for all i∈[1, . . . , M]\j (i.e., for all i≠j) using the least number of samples of the form {θ,r}.

Note that the differences in the dynamics of the neuron types may limit the possible firing rate vectors. (e.g., if all neurons have similar dynamics, selective stimulation at a high rate for only the desired neuron-type may not be possible). Another limitation comes from how rich Θ, and hence [symbol] _{Θ}, is, and thus, in practice, the choice of Θ needs to be made carefully. In some embodiments of the disclosed method, only two neuron types (i.e., M=2) are considered.

The second embodiment is an improvement to the method of the first embodiment. The method of the first embodiment is a non-adaptive data-driven algorithm for determining a pseudo inverse function that will aid in the design of waveforms that elicit desired firing rates in different neuron types. The first embodiment appropriately inverts the forward mapping/relation from waveform parameters within a restricted domain to the firing rate of neuron to obtain a pseudo inverse function, which then can be used to obtain an estimate of a waveform parameter that elicits a firing rate in each neuron type which us close to some desired firing rate. The pseudo inverse function is determined over a restricted domain, which is also estimated by the first embodiment. A limitation of the method of the first embodiment is that the dataset used to estimate the pseudo inverse function and the restricted domain is unrestricted and known a priori and is not changed by the method if the first embodiment.

The method of the second embodiment extends the first embodiment to an adaptive, data driven method for designing selectively stimulating waveforms. To collect new datapoints, the second embodiment uses the first embodiment to extrapolate from existing data points and estimates a waveform corresponding to a higher degree of selectivity Then, the second embodiment refines an adaptive sampling scheme to sample densely in a concentrated region around the estimate of the more selective waveform. This adaptive sampling scheme enables the second embodiment to design waveforms in a data-efficient manner.

The method of the second embodiment, which designs selective waveforms in a data-efficient manner, will now be disclosed. The method iteratively collects data in rounds, where, in each round, N datapoints are collected (except for the first round, wherein data points are uniformly sampled from an unrestricted domain). A data point comprises a set of parameters defining an electrical waveform and the observed neural response. At each round, each data point is used to generate an electronic waveform using an arbitrary waveform generator. The generated waveform is then used to stimulate a brain region from which the corresponding neural response are recorded.

The novelty of the second embodiment is the exploitation of the data collected in previous iterations to estimate data points exhibiting higher selectivity, and then densely sample a data point exhibiting the most selective neural response. By sampling in a region concentrated around more selective data points, the method finds waveforms with a high degree of selectivity by only sampling a few data points.

Denote all of the data collected until iteration t by [symbol] ₜ. Let [symbol] and ŵ(θ) be the estimated pseudo inverse and the corresponding weight mapping for the restricted domain, respectively, estimated by the method of the first embodiment using [symbol] ₜ. Define r_{t,j}^{max} as the most selective firing rate vector for neuron j observed until round t:

$${r}_{t , j}^{max} = arg {max}_{r ∈ {ℛ}_{t}} {r}_{j} s . t . {r}_{i} \leq {r}_{l o w} ∀ i ∈ \{ 1 , … , M \} ∖ j \qquad ( 10 )$$

where:
[symbol] ₜ is the collection of all firing rates collected until round t; and
r_{low} is a pre-specified threshold on the maximum firing rate of all other neuron types.

At iteration t, the method performs two operations: (i) find an estimate [symbol] of a waveform parameter (θ_{d}^{t}) that elicits a more selective firing rate rₛ^{t} than r_{t,j}^{max}. Note that [symbol] being an estimate, is not guaranteed to elicit firing rate rₛ^{t}, but rather something close to it; (ii) sample waveform parameters (i.e., data points) from a region concentrated around [symbol] (the more concentrated the better). rₛ^{t} is defined by increasing the firing rare of neuron j in r_{t,j}^{max} by some positive number δ. In effect, the restricted domain is narrowed to an area around the data point that elicits the most selective neural response (i.e., high firing rate of the selected neuron type and low firing rates of other neuron types). There are two strategies disclosed herein for the narrowing of the restricted domain.

For estimating θₛ^{t}, the second embodiment method uses the first embodiment to obtain [symbol] , which then can be used to obtain the estimate [symbol] of θₛ^{t} by simply plugging rₛ^{t} into [symbol] , (i.e., of [symbol] = [symbol] (rₛ^{t})). In various embodiments, the method uses one of two possible strategies (or a combination of the strategies) to reduce the waveform search space for sampling in a concentrated region around [symbol] .

In a first strategy of the second embodiment, the standard deviation [symbol] of θₛ^{t} is estimated. The standard deviation [symbol] is an indicator of how confident [symbol] is in its estimate of [symbol] . Intuitively, the samples should be selected densely around [symbol] if [symbol] is small and coarsely if [symbol] is large. Hence, instead of sampling the whole domain Θ, the smaller region Bₜ (i.e., the narrowed restricted domain) is sampled:

$${B}_{t} = \{ θ ∈ Θ \vert - ⪯ θ ⪯ + \} \qquad ( 11 )$$

where:
[symbol] denotes element-wise inequality.

For each single parameter θᵢ, sampling uniformly from Bₜ is the equivalent to uniformly sampling the interval |θ_{i,t}^{s}−\hat{σ} _{t,i}, θ_{i,t}^{s}+\hat{σ}_{t,i}, where \hat{θ}_{i,t}^{s} and \hat{σ}_{t,i}, i are i^{th} components of θₜ^{s} and \hat{σ}ₜ, respectively.

In a second strategy of the second embodiment, only θ's that lie in the restricted domain estimated by the first embodiment of the invention at round t are selected (i.e., sampled from Bₜ). The restricted domain is chosen such as to include as many firing rate vectors as possible. Hence, by only considering θ lying in the restricted domain, a significant reduction in the parameter search space can be achieved while ensuring a more selective parameter can still be sampled. To select data points belonging to the restricted domain, realize that [symbol] is approximately 0 for data points not in the restricted domain. A rejection sampling procedure is used, where a θ (sampled from Bₜ) is accepted with probability pₜ(θ)=(\hat{ω}(θ)−ωₘᵢₙ)/(ωₘₐₓ−ωₘᵢₙ). Here, ωₘₐₓ^{t}=max_{θ∈B}_{ₜ} [symbol] (θ) and ωₘᵢₙ^{t}=min_{θ∈B}_{ₜ} [symbol] (θ). Note, pₜ(θ)≈1 for high values of [symbol] (θ) and pₜ(θ)≈0 for low values of [symbol] (θ), thereby encouraging sampling of θ values lying in the restricted domain.

The two strategies may be used alone, however, application of both of the strategies together leads to significant reduction in the region of parameter space being sampled, thereby allowing the method to quickly find selectively stimulating waveform parameters. The method of the second embodiment terminates when either a desired level of selectivity is achieved or the maximum budget for collecting the dataset is reached.

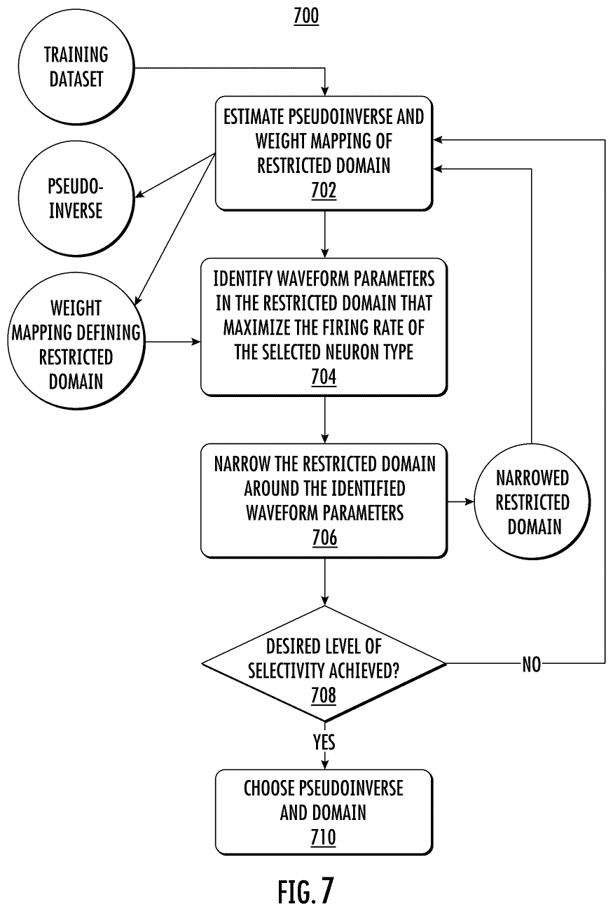

A process 700 implementing the second embodiment is shown in flow chart form in FIG. 7. At 702, a pseudo inverse and restricted domain are estimated in accordance with the first embodiment using a dataset from a restricted domain (except in the first interaction, wherein the dataset is uniformly selected from an unrestricted domain) and producing a pseudo inverse function and a restricted domain. At 704, data points in the restricted domain are sampled to determine the data point producing the neural responses closest to the desired neural response (i.e., the highest firing rate of the selected neuron type and lowest firing rates of other neuron types). At 704, the restricted domain is narrowed using one or both of the first or second strategies. The narrowed restricted domain is then used to estimate a more selective pseudo inverse function and restricted domain in a next iteration of the method. The method continues to iterate at 708, until a desired selectivity is achieved (i.e., the selected neuron type fires at a high firing rate while other neuron types fire at a low rate), or until a maximum computing or energy budget for collecting the dataset is reached.

Both embodiments of the invention are agnostic to the data collection process, but typically, the neural responses are collected through some form of electro-physiological recording. For example, the neural responses can be collected from patch-clamp recordings, EMG responses, EEG, sEEG, or standard Local Field Potentials.

As would be realized by those of skill in the art, the specific examples discussed herein have been provided only as exemplary embodiments and the invention is not meant to be limited thereby. Modifications and variations are intended to be within the scope of the invention, which is given by the following claims:

1. A method comprising:
determining a pseudo inverse function of a forward mapping function between an electrical waveform characterized by one or more data points and a neural response generated by the one or more data points;
determining a restricted domain of data points over which the pseudo inverse function is valid;
sampling data points from the restricted domain to determine a best response data point providing a neural response closest to a desired neural response;
narrowing the restricted domain around the best response data point; and
iterating the method using data points selected from the narrowed restricted domain to generate the pseudo inverse function and the restricted domain in a next iteration of the method.

2. The method of claim 1 wherein each data point represents one or more parameters defining an electrical waveform.

3. The method of claim 1 wherein the desired neural response is a selective firing rate of a particular neuron type.

4. The method of claim 3 wherein the best response data point is the datapoint that increases the firing rate of the particular neuron type by a predetermined number.

5. The method of claim 3 wherein the desired neural response is an increased firing rate of the particular neuron type and a decreased firing rate of other neuron types.

6. The method of claim 3 wherein the iteration is terminated when a desired level of selectivity is achieved or the maximum budget for collecting the data points is reached.

7. The method of claim 2 further comprising:
generating one or more electrical waveforms using data points sampled from the restricted domain; and
recording a neural response of a subject to the application of the electrical waveforms.

8. The method of claim 1 wherein narrowing the restricted domain around the best response data point comprises:
estimating a standard deviation of the best response data point; and
narrowing the restricted domain to data points within a positive or negative standard deviation of the best response data point.

9. The method of claim 1 wherein narrowing the restricted domain around the best response data point further comprises:
estimating a standard deviation of the best response data point; and
narrowing the restricted domain to data points within a positive or negative standard deviation of the best response data point that also lie in the restricted domain.

10. The method of claim 1 wherein, in the first iteration, data points are uniformly sampled from an unrestricted domain.

11. A system comprising:
an electrical waveform generator; and
a device performing electro-physiological recording to record neural responses;
a processor; and
software that, when executed by the processor, performs the functions of:
determining a pseudo inverse function of a forward mapping function between an electrical waveform generated by the electrical waveform generator based on one or more data points and a neural response generated by the one or more data points as recorded by the electro-physiological recording device;
determining a restricted domain of the data points over which the pseudo inverse function is valid;
sampling data points from the restricted domain to determine a best response data point providing a neural response closest to a desired neural response;
narrowing the restricted domain around the best response data point; and
iterating the functions performed by the software using data points selected from the narrowed restricted domain to generate the pseudo inverse function and the restricted domain in a next iteration.

12. The system of claim 11 wherein each data point represents one or more parameters defining an electrical waveform.

13. The system of claim 11 wherein the desired neural response is a selective firing rate of a particular neuron type.

14. The system of claim 13 wherein the best response data point is the data point that increases the firing rate of the particular neuron type by a predetermined number.

15. The system of claim 13 wherein the desired neural response is an increased firing rate of the particular neuron type and a decreased firing rate of other neuron types.

16. The system of claim 13 wherein the iteration is terminated when a desired level of selectivity is achieved or the maximum budget for collecting the data points is reached.

17. The system of claim 12 further comprising:
generating one or more electrical waveforms using the electrical waveform generator using data points sampled from the restricted domain; and
recording, using the electro-physiological recording device, a neural response of a subject to the application of the electrical waveforms.

18. The system of claim 11 wherein narrowing the restricted domain around the best response data point comprises:
estimating a standard deviation of the best response data point; and
narrowing the restricted domain to data points within a positive or negative standard deviation of the best response data point.

19. The system of claim 11 wherein narrowing the restricted domain around the best response data point further comprises:
estimating a standard deviation of the best response data point; and
narrowing the restricted domain to data points within a positive or negative standard deviation of the best response data point that also lie in the restricted domain.

20. The system of claim 11 wherein, in the first iteration, data points are uniformly sampled from an unrestricted domain.
